(19) United States
(12) Patent Application Publication
(10) Pub. No.: US 2004/0249283 A1
(43) Pub. Date: Dec. 9, 2004

(54) Method and apparatus for breast imaging utilizing ultrasound

(76) Inventors: Edward Kantorovich, Rehovot (IL); Avishai Shavitt, Shoham (IL)

Correspondence Address: William H Dippert, Reed Smith, 29th Floor, 599 Lexington Avenue, New York, NY, 10022-7650, US

(21) Appl. No.: 10/477,310

(22) PCT Filed: May 23, 2001
(86) PCT No.: PCT/IL01/00473

(30) Foreign Application Priority Data
May 9, 2001 (IT) 143060

(51) Int. Cl. A61B 8/00

(52) U.S. Cl. 600/442

(57) ABSTRACT

A method of determining accurate values of acoustic parameters of tissues, comprising: transmitting first acoustic energy through the tissue along a path in a first direction; acquiring a first reflection signal generated by said tissue along said path from the transmitted first acoustic energy; transmitting second acoustic energy through the tissue along the path in a second direction opposite from the first direction; acquiring a second reflection signal generated by said tissue along said path from the transmitted second acoustic energy; and generating one or more of values of attenuation along the path, velocity of acoustic energy along the path, acoustic impedance of materials along the path and reflection along the path, responsive to both the first and second acquired reflection signals.

### FIELD OF THE INVENTION

The invention relates to imaging using ultrasound and in particular to breast imaging using ultrasound.

### BACKGROUND OF THE INVENTION

Ultrasound imaging is a well known. In general, a pulse of ultrasound is emitted by a transducer into a human body structure. Changes in impedance of the body structures, along the path of the ultrasound waves set up in the body, cause a portion of the beam to be reflected or diffracted. A receiving transducer (often the same transducer as is used for transmission) detects the reflected waves. Further structures in the path of the wave reflect additional waves to the receiving transducer. These waves are distinguished from the first reflection by the time at which they reach the transducer, with the waves from more interior impedance interfaces reaching the receiving transducer later than those from interfaces closer to the sending transducer.

If the ultrasound waves are formed in a beam and the beam is scanned across the region to be studied, a two or three-dimensional image of the (impedance of) the internal structure of the region can be determined.

One of the problems in determining the impedance of the internal structures is that of compensating for attenuation. As is well known, the reflection at an interface is dependent on the impedance on the sides of the interface. However, by the time the incident wave reaches the interface it is attenuated by intermediate structures in its path. The reflected beam is similarly attenuated. Thus, the impedance levels implicit in the reflected beam can only be estimated by assuming the attenuation along the path. At best, this results in only an approximation of the impedance of the bodies along the beam path and makes it automated analysis difficult if not impossible.

U.S. Pat. No. 5,984,570 the disclosure of which is incorporated by reference, describes an automated diagnostic method for ultrasound. However, this reference (and the method described) suffers from the lack of information on the actual attenuation of the intervening structures.

### SUMMARY OF THE INVENTION

A broad aspect of some embodiments of the invention is concerned with production of an ultrasound image in which the effects of intervening attenuation are substantially neutralized.

In accordance with an exemplary embodiment of the invention ultrasound reflection signals are generated along a same path in opposite directions. These two reflection signals (referred to herein as first and second reflection signals) contain substantially the same information with respect to the impedance interfaces along the path. However, for a given impedance interface, they are differently affected by the attenuation in their path, since signals from a same interface follow different portions of the path for the opposite directions. In addition, the sense of the reflections (plus or minus) and the time scale of the reflections are reversed.

In accordance with an embodiment of the invention, the time scale of the reflections of one of the first and second reflection signals is inverted about an easily discernible point in both signals. Such a point could be a strongly reflecting interface in the path or the outside edge of the portion being imaged. This will result in features (impedance interfaces) appearing at the same time.

However, the amplitude of these features will be different, for two reasons. The first of these is that the sense of the reflection at any given impedance interface will be different for the two signals. However, since for a given interface, the reflection will have an opposite sign in the two signals, the product will always be negative. Positive values, which are believed to be noise, are optionally truncated. Alternatively, one of the signals may be sign inverted. Alternatively, the absolute value of the reflection can be used in processing the signals. It is noted that the lowest noise is probably achieved when the signals are multiplied without pre-inversion or taking the absolute value.

The second reason is that the two signals have different attenuation paths for the two reflection signals. Furthermore, the attenuation difference for each interface in the path is different. In accordance with an exemplary embodiment of the invention, the amplitudes of the two signals (one of which is time reversed and one of which is amplitude (or phase) reversed) are multiplied by each other. This results in a signal in which the effects of intervening attenuation are neutralized. The difference between the impedances of the materials, on either side of the interface, is directly derivable from the multiplied signal using transmission line equations, for example.

When the signals are multiplied, the general effect is to apply an attenuation to the reflections from all of the interfaces that is equal to the total attenuation of the path. The advantage is that the resultant signal allows for the determination of the magnitude of change in impedance at the interfaces, without additional correction for attenuation. It should be understood that the sign of the change (higher-to-lower or lower-to-higher) of impedance is lost during the multiplication process. However, the sign information can be easily derived from the first and second reflection signals, in which the sign of the reflection is not affected by the attenuation in the path.

The above-described method results in a signal to noise for the interface steps that is intermediate the best and worst signal to noise ratios for the first and second reflectance signals. It has been found that the signal to noise over the entire length of the path is slightly lower than the signal to noise at the center of the path for either signal. It may be desirable, to tailor the frequency used so that the signal to noise level is above a desired value. Moreover, in some circumstances, for regions on either end of the path, it may be desirable to estimate the attenuation in the path and use the higher signal to noise ratio of one of the signals to determine the reflection. However, this is not generally necessary.

Since the above method neutralizes the effect of intermediate attenuation on the determination of impedance steps, it may, under some circumstances, allow the use of a higher frequency irradiation. Such higher frequencies were previously considered useful only for very shallow depths, due to the increase in attenuation with frequency.

An aspect of the invention deals with imaging apparatus for ultrasound imaging of the breast.

In an exemplary embodiment of the invention, the apparatus includes a liquid filled container into which the breast of a patient, lying face down in a prone position, is placed. Means are provided for acquiring reflection type ultrasound signals along many paths through the breast. Further, the acquisition is generally designed to enable acquisition of reflection data from opposite directions. In some embodiments of the invention, the path in the liquid is minimal to avoid attenuation in the liquid (and subsequent reduction in signal to noise). However, this is usually not a problem.

In some embodiments of the invention, the liquid comprises a liquid sterilizing agent, as known in the art, which provides a generally low loss path that matches the acoustic properties of the breast. It should be noted that since the impedance and attenuation of the liquid is generally known, the first reflection (between the liquid and the breast), allows for generally simple determination of the impedance of the surface portion of breast tissue.

Various means for acquiring the first and second reflection signals are possible. For example, a circular array of transducers (transmit/receive) may be placed around the breast, facing toward the center of the circle. Alternatively, the array is not circular. If the density of the transducers is high enough to provide the image pixel density required, the array could be stationary, or at least non-rotating. In general, the transducers are generally arranged in pairs, with the elements of each pair transmitting along a same path in different directions.

As indicated the array need not be circular. One example of a suitable array is a dual linear array of elements, with corresponding elements in each of the dual arrays facing each other. This array system has the advantage that the density of paths is the same throughout the breast.

Optionally, each transducer is focused or at least collimated, so that the spatial resolution of the system is increased.

A second means for acquiring the first and second reflection signals utilizes an array or arrays that rotate about the breast, acquiring images from many directions. Again the transducers can be arranged in a linear or circular array, or in any form that allows for acquisition of reflections along paths, in opposite directions.

While single fixed direction transducers are described above, it should be understood that electronically steerable transducers could also be used. Such transducers are capable of providing acoustic reflection data along a multitude of paths. While only paths that have a paired set of transducers at both ends can be utilized in the method described above, the other paths can also yield useful information regarding the structure of the object being probed.

In one embodiment of the invention, the array is movable with respect to its axis (for a circular array) or perpendicular to their long edges (for linear arrays) in order to allow for complete coverage of the volume of the object (for data acquisition) and three dimensional reconstruction of the object. Alternatively, a cylindrical array or two opposed planar arrays are used. If the array transducers are steerable, paths between transducers on different “levels” of the arrays can be combined, as described above. Similarly, for steerable array elements, paths between non-geometrically opposing transducers can be paired.

It should be understood that there is no requirement that the first and second reflection signals be acquired without moving the transducers. Thus, in principle, a single transducer, which is positionable at points on a cylinder surrounding the breast may be used. First and second reflection signals for any path would then be acquired when the transducer is positioned at opposite ends of the path. An alternative structure is a linear array parallel to the axis of the breast. Rotating the array about an axis parallel to that of the breast allows for the acquisition of a plurality of reflection signals for parallel paths, at a single position of the transducer array. Alternatively, a single planar array rotated about such an axis allows for the acquisition of a three dimensional array of first or second reflection signals. In principle, only two oppositely placed positions of the array are necessary for determining the structure of the entire object. However, it may be desirable to provide a number of crisscrossing paths and to combine the data to improve the quality and reliability of the data. Other possible configurations, such as half circular or half cylindrical will occur to a person of skill in the art.

While the number of transducers can be reduced in this manner, it should be understood that the required mechanical motion does reduce the speed with which an examination can be performed.

Planar arrays (or linear arrays that scan in a planar manner) have the advantage that the resolution throughout the breast is the same. Circular/cylindrical geometries have the advantage of smaller size and simplicity of construction.

A third aspect of some embodiments of the invention is concerned with aids to diagnosis. As is well known, the objective of breast imaging is the disclosure and characterization of anomalies. For example, ultrasound is generally capable of distinguishing between cystic lesions and solid lesions and, to some extent between malignant and non-malignant lesions. Applicants have discovered that with the information (one or more of absolute impedance, improved attenuation and velocity values) provided by the above methods, it is possible to more precisely determine the actual velocities and attentions of the tissues which should result in more precise tissue differentiation.

There is thus provided, in accordance with an exemplary embodiment of the invention, a method of determining accurate values of acoustic parameters of tissues, comprising:

transmitting first acoustic energy through the tissue along a path in a first direction;

acquiring a first reflection signal generated by said tissue along said path from the transmitted first acoustic energy;

transmitting second acoustic energy through the tissue along the path in a second direction opposite from the first direction;

acquiring a second reflection signal generated by said tissue along said path from the transmitted second acoustic energy; and

generating one or more of values of attenuation along the path, velocity of acoustic energy along the path, acoustic impedance of materials along the path and reflection along the path, responsive to both the first and second acquired reflection signals.

In an embodiment of the invention, generating includes generating values of reflection along the path. Optionally, generating reflections along the path comprises generating a value associated with values of the first and second signals at a given location on the path. Optionally, generating a value associated with a given location comprises:

multiplying, a value derived from the first signal, corresponding to a point on the path, by the value derived from the second signal, corresponding to the same point on the path, for at least one given location along the path.

Optionally, the at least one location comprises a plurality of locations along the path. Optionally, the plurality of locations along the path comprises a spaced array of locations along the entire path. Optionally, the plurality of locations comprises locations corresponding to transitions in the acoustic impedance along the path.

In an embodiment of the invention, generating includes generating values of attenuation along the path. Optionally, generating of values of attenuation includes:

determining values of reflections along the path; and

generating the values of attenuation from said values of reflection and one of the first and second reflection signals.

Optionally, generating the values of attenuation comprises comparing the amplitude value of a reflection as it is present in the reflection signal with the actual reflection value and determining the attenuation based on the difference between the compared values.

Optionally, the method includes providing a total attenuation along the path and utilizing the provided total attenuation to generate the one or more values.

Optionally, generating of values of attenuation includes:

determining values of reflections along the path;

determining a total attenuation of acoustic waves along the path; and

generating the values of attenuation from said values of reflection and said total attenuation.

Optionally, the total attenuation is determined from a measurement of attenuation along the path. Optionally, the attenuation is measured by acquiring a signal at one end of the path generated by an acoustic wave that is generated at the other end of the path and travels along the path.

Optionally, the method includes determining a total transit time of the acoustic energy along the path and utilizing the determined total transit time to generate the one of more values.

In an embodiment of the invention generating includes generating acoustic velocity values along the path. Optionally, generating acoustic velocity values along the path comprises estimating or determining the acoustic velocity in normal tissue along the path. Optionally, determining the acoustic velocity in normal tissue comprises measuring a transit time of acoustic waves along an auxiliary path including only normal tissue and optionally a material of known acoustic properties.

In an embodiment of the invention, the method includes:

determining a transit time of an acoustic wave along the path;

reducing the determined transit time by a transit time for normal tissue and optionally materials of known velocity in the path;

determining a total length for the path;

reducing the total length by a length of normal tissue and optionally other materials of known length in the path; and

determining the velocity in abnormal tissue the path from the reduced transit time and reduced length. In an embodiment of the invention, the values include acoustic impedance values along the path. Optionally, generating acoustic impedance values along the path comprises:

providing a known impedance for material situated along a portion of the path;

determining the amplitude and sign of reflections along the path; and

determining the impedance change at successive locations of reflection along the path, based on the known impedance and the signs of reflections along the path.

Optionally, the tissue comprises a breast.

In an embodiment of the invention, the method includes acquiring said first and second signals along a plurality of paths in a plane and providing said one or more values along a plurality of locations along a plurality of paths in said plane. Alternatively or additionally, the method includes acquiring said first and second signals along a plurality of non-coplanar paths in volume and providing said one or more values along a plurality of locations along a plurality of paths in said volume.

Optionally, the method includes providing an image of said one or more values.

There is further provided, in accordance with an embodiment of the invention, apparatus for ultrasound imaging, comprising:

at least one pair of acoustic transducers places on opposite sides of a region, said transducers forming a path therebetween;

an acoustic generator for activating said transducers to produce acoustic waves traveling along said path; and

an acoustic detector for receiving at least reflection signals from said region in response to said produced acoustic waves.

In an embodiment of the invention, the at least one pair of transducers comprises a plurality of oppositely spaced transducers positioned along the circumference of a ring. Optionally, the apparatus includes a plurality of axially spaced rings of transducers. Optionally, the apparatus includes means for axially displacing the ring, so as to provide coverage of a volume of the region.

In an embodiment of the invention, the at least one pair of transducers comprises a plurality of oppositely spaced transducers positioned along straight lines. Optionally, the invention includes a plurality said linearly arrayed transducers, forming a pair of planar arrays of transducers, said arrays providing coverage of a volume of the region. Optionally, the apparatus includes means for rotating the pair of transducers about an axis between the transducers in a pair.

There is further provided, in accordance with an embodiment of the invention, apparatus for ultrasound imaging, comprising:

at least one acoustic transducers facing into a region at a first position;

means for positioning the transducer to a second position on an opposite side of the region, said transducers at said positions forming a path therebetween;

an acoustic generator for activating said transducers to produce acoustic waves traveling along said path; and

an acoustic detector for receiving at least reflection signals from said region in response to said produced acoustic waves.

In an embodiment of the invention, the at least one transducer comprises a plurality of spaced transducers positioned along a straight line. Optionally, the apparatus includes a plurality said linearly arrayed transducers, forming a planar array of transducers, said array providing coverage of a volume of the region.

Optionally, the apparatus includes a signal combiner for receiving said signals and combining said signals received from transducers oppositely placed on the path to provide acoustic impedance values along the path.

### BRIEF DESCRIPTION OF FIGURES

Exemplary embodiments of the invention are described in the following description, read in with reference to the figures attached hereto. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a schematic representation of a breast imaging system in accordance with an exemplary embodiment of the invention;

FIGS. 2A-2E show various configurations of transducers, in accordance with an embodiment of the invention;

FIGS. 3A-3C illustrate the acquisition and processing of first and second reflection signals, in accordance with an embodiment of the invention; and

FIG. 4 is a flow chart of a representative method of determining acoustic velocity, in accordance with an embodiment of the invention.

### DETAILED DESCRIPTION OF EXEMPLARY EMBODIMENTS

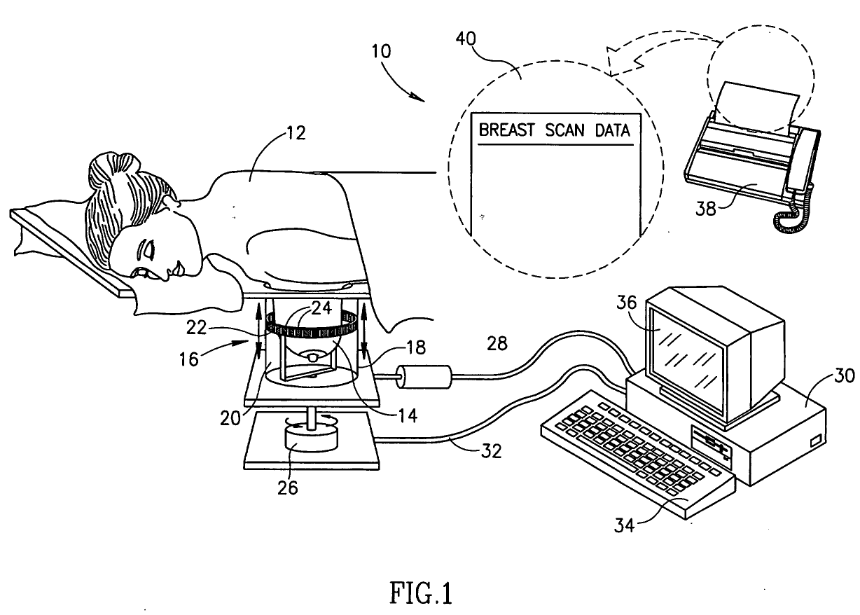

FIG. 1 shows an exemplary system 10 for performing acoustic breast scanning in accordance with an embodiment of the invention.

A patient 12, is shown lying in a downward prone position, with a breast 14 protruding into an input section 16 of scanning system 10. While breast imaging is shown, the adaptation of the methods and apparatus described herein to imaging of other extremities or of the trunk of a patient, for example the abdomen or chest.

Input section 16 comprises a container 18, filled with a liquid 20 (for example water with an added sterilizing agent), such that as much as possible of breast 14 is immersed in liquid 20. A circular array 22 surrounds breast 14 and is movable along an axis generally parallel to that of the breast so that the breast can be scanned along its entire length. Array 22 is shown, for clarity, as having a relatively small number of large, spaced transducers 24. However, smaller, more closely spaced transducers may be used. The transducers may use a common element for both irradiation and detection or separate detector and transmitter elements may be used.

A motor system 26 optionally provides for movement of circular array 22 in a vertical direction. This allows for the coverage of the breast along its entire length. It should be understood that due to physical constraints, it may not be possible to image the portion of the breast that is adjacent to the chest wall, using the imaging method described herein. However, if transducers 24 are upwardly steerable, at least by a small amount, reflection images in one direction can be acquired into the chest wall. Alternatively, a regular ultrasound examination is performed either before or after the examination described herein to image the breast near the chest wall.

If the desired resolution is greater than is achievable by the spacing of the transducers, a small amount of rotation, at each level, is also provided so that rotationally intermediate positions of the transducers and acquisition of data at a higher resolution are possible.

Electrical signals for generating acoustic irradiation and for transmitting reflection signals acquired by the transducers are transmitted via a cable 28 or other wireless or wired communication means from and to a controller or computer 30. Controller 30 also controls the operation of motor system 26 via a cable 32 or other wired or wireless communication means. In exemplary systems, computer 30 generates only control signals, with the actual acoustic or motor power voltages being generated at or near the devices being controlled.

Exemplary systems 10 also include a user interface 34, exemplified by a keyboard. However, such an interface may comprise a joystick, trackball, mouse, touch-screen or any other input device known in the art. A display 36 is also generally provided, both for viewing images or other data and as part of the input to the system. Such displays are well known in the art. A hard copy device 38 may be provided to produce hard copies 40 of images or of a diagnostic output as described below.

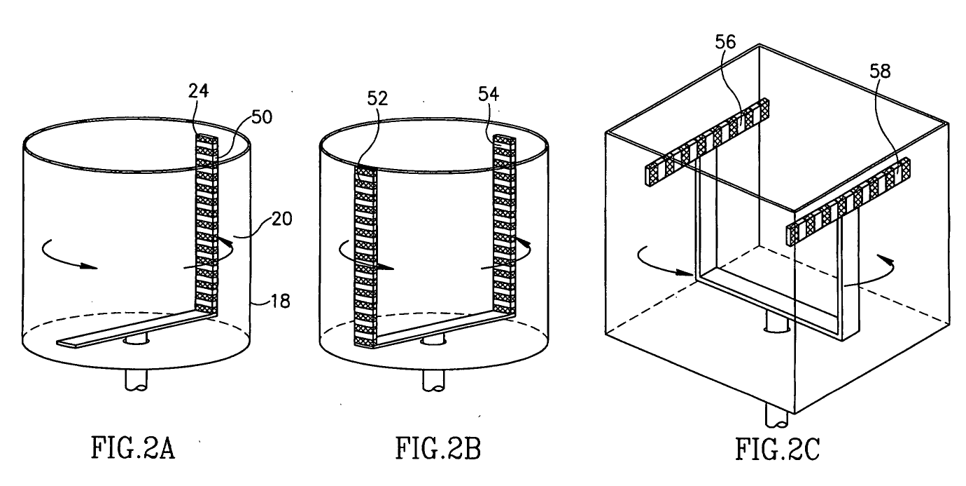

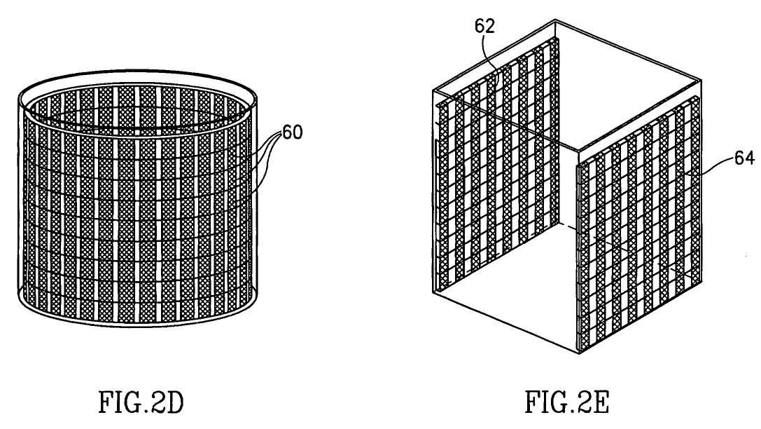

FIGS. 2A-2E show various alternative configurations of transducer arrays, useful in the practice of various embodiments of the invention. FIG. 2A shows a linear array 50 having a long direction substantially parallel to an axis of the breast. The entire breast can be scanned by rotation the array about the breast axis (or a substantially parallel axis as shown). Such rotation can be provided by a motor or the like as is well known in the art.

FIG. 2B shows a pair of linear arrays 52 and 54, structurally similar to array 50, situated on opposite sides of the breast.

FIG. 2C shows a pair of linear arrays 56 and 58 arranged substantially perpendicular to the breast axis and optionally rotatable, as shown. In order to image the entire breast the array pair is translated along the direction of the breast axis to acquire a “slice” image at each position along the axis. Optionally, multiple views are acquired at each height. It should be understood that a single array 56 could be used. In either event, if such an array is used, a larger sized container 18 is required (as compared to the arrays of FIG. 2A, 2B or 2D) to accommodate a same size breast. If rotation is desired or required, container 18 must be large enough (as shown) to accommodate such rotation. If no rotation is desired, the container can be smaller. However, it will still be larger than a comparable device with cylindrical geometry. Axial and rotational motion is provided by a motor or actuator or a combination of motors and actuators.

FIG. 2D shows a cylindrical array 60, for which, if the array pitch is low enough, no movement is necessary. It should be understood that while such arrays are generally more expensive than simple linear arrays. They do have the advantage that no motion is required and that the examination can proceed more quickly.

FIG. 2E shows a pair of planar arrays 62 and 64. Again, if the array pitch is low enough, no movement is necessary. It is noted that planar arrays give a more uniform resolution over the scanned volume, but require more transducers and a larger volume container. As above, a single planar array, rotated about the breast can also be used.

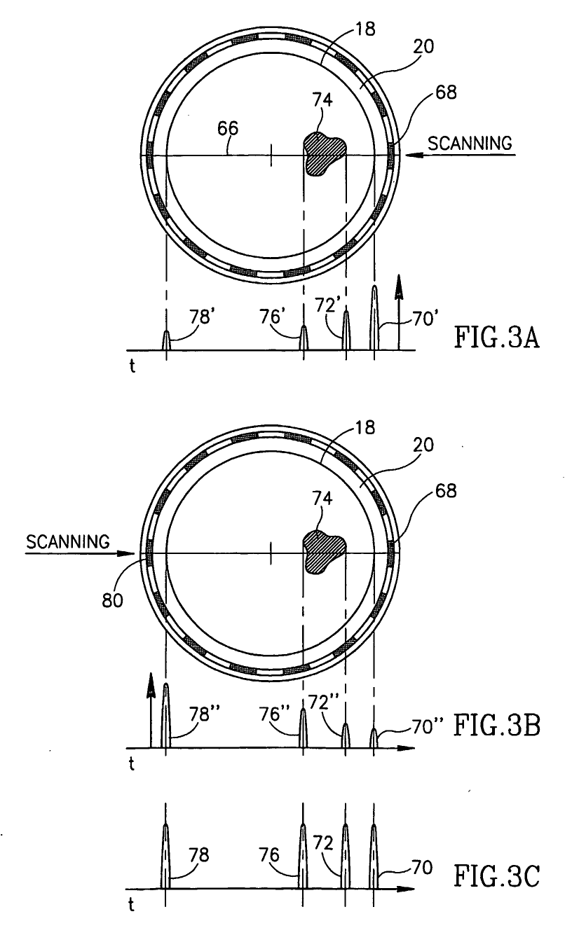

FIGS. 3A-3C illustrate, in simplified schematic form, the process utilized in neutralizing the effects of attenuation on the signals generated by interfaces of different materials along a line 66. While the process is shown, for simplicity as being along a line, it is understood that an acoustic beam generated by the transducers is generally at least partially focused along its length so that the reflections are actually area reflections. This may, as is well known in the art, cause a certain amount of smearing of the reflection. However, unless such smearing is very large, it does not effect the present analysis.

The upper portion of FIG. 3A shows a scan line from a transducer 68. The lower portion of the FIG. 3A shows the reflection signal as acquired by transducer 68. The reflection contains four components. A first component 70′ is the reflection between the impedance interface of surrounding liquid 20 and breast 18. For simplicity of presentation, the impedance ratio at each interface is the same, although, in general, this is not the case. However, as seen in FIG. 3A, component 70′ is comparatively large. This is the case, since for the direction shown there is only a small amount of attenuation of the signal. A second component 72′ represents the reflection from the impedance interface between the normal breast tissue and an anomaly (shown here as a solid growth) 74. This anomaly causes a reflection from the interface. Although (in proportion to the impinging acoustic wave) this reflection is the same as that from the liquid/breast interface, component 72′ is smaller than component 70′, due to the longer path the acoustic wave travels to an from the anomaly and the additional attenuation it suffers.

A third component 76′ is the reflection from the interface between the anomaly and the normal breast tissue, as the wave leaves anomaly 74. In principle, this reflection should be (proportionally) equal and opposite in sign to that of component 72′. However, due to attenuation, it is smaller.

It should be noted that all the reflections are shown as being in the same direction. While the acoustic reflections can be positive or negative depending on the sign of the impedance step, in the practice of the invention, any interface will cause an oppositely signed transition in the two reflection signals. Since for a given interface, the reflection will have an opposite sign in the two signals, the product will always be negative. Positive values, which are believed to be noise, are optionally truncated. Alternatively, one of the signals may be sign inverted. Alternatively, the absolute value of the reflection can be used in processing the signals. It is noted that the lowest noise is probably achieved when the signals are multiplied without pre-inversion or taking the absolute value. For simplicity, the signals are shown as their absolute value.

Fourth component 78′ represents the reflection as the wave leaves breast 20 and enters liquid 18.

FIG. 3B shows a similar situation, in which the acoustic wave is transmitted by a transducer 80 (which also receives the reflected signals) along path 66, but in an opposite direct from that of FIG. 3A. In this case, all of the reflections of FIG. 3A are present (shown as double primed numbers), but in reverse time order. In addition, the sense of the reflections is reversed (not shown, as described above). As in FIG. 3A, the closer reflections produce stronger signals, since they suffer less attenuation.

FIG. 3C shows the product of the signals shown in FIGS. 3A and 3B (shown to a different scale). As indicated above, the interface impedance ratios were assumed the same for all the interfaces. When the two signals are multiplied the effect of the attenuation is compensated for and all the reflections are the same size. This is true since if the total attenuation is A, then for every point x along the path, the attenuation can be expressed as A=A(left, x)*A(right, x). Thus, when the two signals are multiplied, the result is not a function of the position of the anomaly, but only on its interface ratio. It may however, be a function of the overall attenuation. In order to remove this effect, the total attenuation either estimated or more preferably determined by measurement of attenuation of an acoustic wave from one end of the path to the other. The signals derived by the above multiplication are then optionally normalized by dividing them by “A” and taking their square root.

Based on the values of reflection, a determination of the impedance of the tissue as a function of position is made, in accordance with an embodiment of the invention.

Since the breast tissue is surrounded by water, whose impedance is known, the reflection at the interface between the breast tissue and the water can be used to determine the impedance of the surface tissue of the breast. This value of impedance is used to determine the value of impedance following the next interface causing a reflection. This process is repeated until the entire breast is traversed.

Similarly, attenuation of the tissues can also be determined. For each reflected signal, the reduction of the amplitude is cause by two components. One of these is the reflections from interfaces. The second component is the attenuation of the signals. Using the accurate values of reflection derived above and assuming (for a first estimate, at least) that the velocity is constant within the breast (and known in the water surrounding the breast) the distance to each interface can be estimated. This velocity can be further refined as described below. This provides a function (utilizing either of the reflection signals) in which the actual amplitude of the reflections is known and the distance is known. The attenuation of the intervening tissues is then derivable as the difference between the expected reflection amplitude and the actual reflection amplitude caused by the interfaces. The effect of distance, which, in principle reduces the signal amplitude by a factor of R⁴, where R is the total path length, is basically neutralized by focusing the beam. However, remaining effects of spreading (or focusing) of the beam may be taken into account.

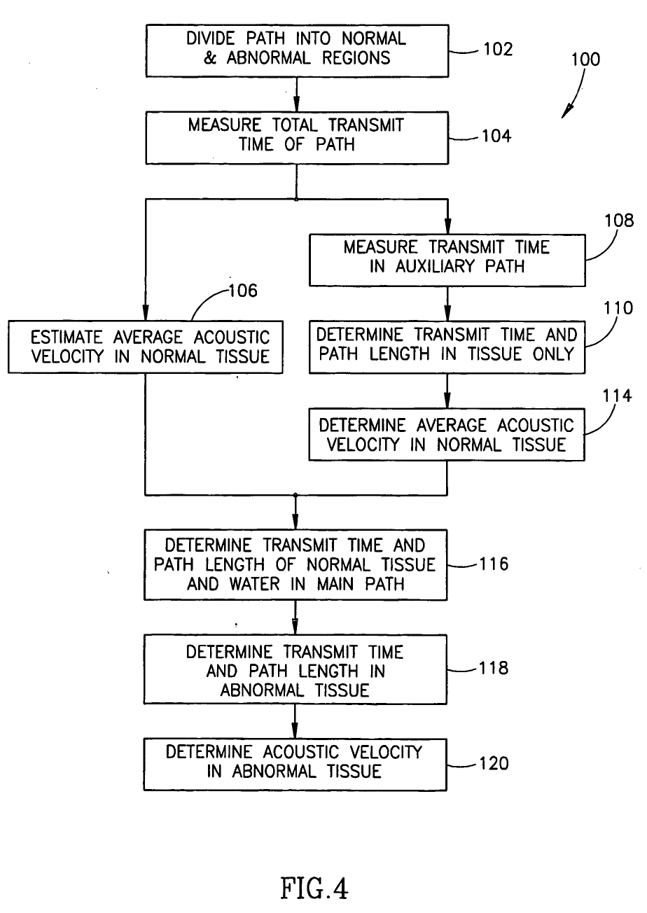

FIG. 4 is a flow chart 100 of a method of determining the acoustic velocity along the path, as a function of position along the path.

At 102, the reflection signal is divided into regions of “abnormal” impedance and regions of “normal” impedance. Experience has shown that regions of high impedance (which generally denote anomalies of interest) have much higher velocities than normal tissue.

The total transit time over the path is measured (104), for example, by measuring the time it takes for an acoustic signal to pass from a transducer (as a transmitter) on one side of the path to a transducer (as a receiver) on the other side of the path.

The transit time for the normal tissues is estimated (106) or measured. It can be estimated from the general knowledge of acoustic velocities in normal tissues being about 1540 m/sec.

Alternatively, the actual velocity can be better estimated by measuring the transit time along an auxiliary path that includes only normal tissue (108). The auxiliary path will include water surrounding the normal tissue and breast tissue (assumed to have a constant velocity). At 110 the transit time of the acoustic wave in the surrounding water is computed and subtracted from the total transit time to give the transit time in tissue only. The length of the path in the breast tissue is determined by subtracting the length of the path in the water (determined from the known acoustic velocity in water and the transit time in water) from the total path length (known from the system geometry). The tissue path length is divided by the tissue transit time (114) to determine the average acoustic velocity in normal tissue.

Returning to the main path, the transit time (and length) of the normal tissue and water in the main path is determined (116). The transit times are determined directly from the reflection signal. The distances can be determined by multiplying the transit times by the respective estimated or determined velocities. The transit times and distances for the normal tissue and water are subtracted from the total transit time and distance for the main path to determine the distance and transit time for the tissue path lengths that exhibit “abnormal” impedance (118). The velocity in the abnormal tissue (120) can be determined by dividing the length of the path in the issue by the transit time in the tissue.

In some cases more than one type of anomalous tissue is present. In such case, it may be possible to determine the velocities utilizing paths that contain only one of the anomalies and normal tissue. The thus determined velocities can then be utilized to refine the size determinations of an anomaly.

While the above method has been described with respect to determining the velocity in “normal” and “abnormal” tissue, it can also be used to differentiate the different velocities of different types of normal tissue.

It should be noted that total transit time and total velocity are described as being measured utilizing transducers on opposite sides of the path (FIGS. 2B-2E). However, it should be noted that these parameters can also be determined (if less well) utilizing a detector on one side of the breast (FIG. 2A). The total path attenuation and transit time are determined from the attenuation and delay time of the reflected waves from the farthest object, for example, the far wall of the container (which may be made reflecting for this purpose). This reflection represents a wave that has traveled twice along the path.

In some of the embodiments described above, values of attenuation, velocity and interface reflection may be derived, for the same voxel of tissue or the same interface, from signals along a plurality of paths. The value chosen for the characteristic(s) being studied may be based on an average of the derived values or by using only the value from a trace with the highest signal to noise. Alternatively, the value chosen may be a weighted average of the derived values.

As is well known, the objective of breast imaging is the disclosure and characterization of anomalies. For example, ultrasound is generally capable of distinguishing between cystic lesions and solid lesions and, to some extent between malignant and non-malignant lesions. Applicants have discovered that with the additional information (absolute impedance) provided by the above methods, it is possible to more precisely determine the actual velocities and attentions of the tissues. Since present methods, are based on relatively imprecise values of these parameters, more precise tissue differentiation, should be possible.

The present invention has been described using non-limiting detailed descriptions of exemplary embodiments thereof that are provided by way of example and that are not intended to limit the scope of the invention. Variations of embodiments of the invention, including combinations of features from the various embodiments will occur to persons of the art. The scope of the invention is thus limited only by the scope of the claims. Furthermore, to avoid any question regarding the scope of the claims, where the terms “comprise,” “comprising,” “include,” “including” or the like are used in the claims, they mean “including but not necessarily limited to”.

1. A method of determining accurate values of acoustic parameters of tissues, comprising:
transmitting first acoustic energy through the tissue along a path in a first direction;
acquiring a first reflection signal generated by said tissue along said path from the transmitted first acoustic energy;
transmitting second acoustic energy through the tissue along the path in a second direction opposite from the first direction;
acquiring a second reflection signal generated by said tissue along said path from the transmitted second acoustic energy; and
generating one or more of values of attenuation along the path, velocity of acoustic energy along the path, acoustic impedance of materials along the path and reflection along the path, responsive to both the first and second acquired reflection signals.

2. A method according to claim 1 wherein generating includes generating values of reflection along the path.

3. A method according to claim 2 wherein generating reflections along the path comprises generating a value associated with values of the first and second signals at a given location on the path.

4. A method according to claim 3 wherein generating a value associated with a given location comprises:
multiplying, a value derived from the first signal, corresponding to a point on the path, by the value derived from the second signal, corresponding to the same point on the path, for at least one given location along the path.

5. A method according to claim 4 wherein the at least one location comprises a plurality of locations along the path.

6. A method according to claim 5 wherein the plurality of locations along the path comprises a spaced array of locations along the entire path.

7. A method according to claim 5 wherein the plurality of locations comprises locations corresponding to transitions in the acoustic impedance along the path.

8. A method according to claim 1 wherein generating includes generating values of attenuation along the path.

9. A method according to claim 8 wherein generating of values of attenuation includes:
determining values of reflections along the path; and
generating the values of attenuation from said values of reflection and one of the first and second reflection signals.

10. A method according to claim 9 wherein generating the values of attenuation comprises comparing the amplitude value of a reflection as it is present in the reflection signal with the actual reflection value and determining the attenuation based on the difference between the compared values.

11. A method according to claim 1 and also including providing a total attenuation along the path and utilizing the provided total attenuation to generate the one or more values.

12. A method according to claim 8 wherein generating of values of attenuation includes:
determining values of reflections along the path;
determining a total attenuation of acoustic waves along the path; and
generating the values of attenuation from said values of reflection and said total attenuation.

13. A method according to claim 11 wherein the total attenuation is determined from a measurement of attenuation along the path.

14. A method according to claim 13 wherein the attenuation is measured by acquiring a signal at one end of the path generated by an acoustic wave that is generated at the other end of the path and travels along the path.

15. A method according to claim 1 and including determining a total transit time of the acoustic energy along the path and utilizing the determined total transit time to generate the one of more values.

16. A method according to claim 1 wherein generating includes generating acoustic velocity values along the path.

17. A method according to claim 16 wherein generating acoustic velocity values along the path comprises estimating or determining the acoustic velocity in normal tissue along the path.

18. A method according to claim 17 wherein determining the acoustic velocity in normal tissue comprises measuring a transit time of acoustic waves along an auxiliary path including only normal tissue and optionally a material of known acoustic properties.

19. A method according to claim 16 comprising:
determining a transit time of an acoustic wave along the path;
reducing the determined transit time by a transit time for normal tissue and optionally materials of known velocity in the path;
determining a total length for the path;
reducing the total length by a length of normal tissue and optionally other materials of known length in the path; and
determining the velocity in abnormal tissue the path from the reduced transit time and reduced length.

20. A method according to any claim 1 wherein the values include acoustic impedance values along the path.

21. A method according to claim 20, wherein generating acoustic impedance values along the path comprises:
providing a known impedance for material situated along a portion of the path;
determining the amplitude and sign of reflections along the path; and
determining the impedance change at successive locations of reflection along the path, based on the known impedance and the signs of reflections along the path.

22. A method according to claim 1, wherein the tissue comprises a breast.

23. A method according to claim 1 and including acquiring said first and second signals along a plurality of paths in a plane and providing said one or more values along a plurality of locations along a plurality of paths in said plane.

24. A method according to claim 1 and including acquiring said first and second signals along a plurality of non-coplanar paths in volume and providing said one or more values along a plurality of locations along a plurality of paths in said volume.

25. A method according to claim 23 and including providing an image of said one or more values.

26. Apparatus for ultrasound imaging, comprising:
at least one pair of acoustic transducers places on opposite sides of a region, said transducers forming a path therebetween;
an acoustic generator for activating said transducers to produce acoustic waves traveling along said path; and
an acoustic detector for receiving at least reflection signals from said region in response to said produced acoustic waves.

27. Apparatus according to claim 26 wherein said at least one pair of transducers comprises a plurality of oppositely spaced transducers positioned along the circumference of a ring.

28. Apparatus according to claim 27 and including a plurality of axially spaced rings of transducers.

29. Apparatus according to claim 27 and including means for axially displacing the ring, so as to provide coverage of a volume of the region.

30. Apparatus according to claim 26 wherein said at least one pair of transducers comprises a plurality of oppositely spaced transducers positioned along straight lines.

31. Apparatus according to claim 30 and including a plurality said linearly arrayed transducers, forming a pair of planar arrays of transducers, said arrays providing coverage of a volume of the region.

32. Apparatus according to claim 26 and including means for rotating the pair of transducers about an axis between the transducers in a pair.

33. Apparatus for ultrasound imaging, comprising:
at least one acoustic transducers facing into a region at a first position;
means for positioning the transducer to a second position on an opposite side of the region, said transducers at said positions forming a path therebetween;
an acoustic generator for activating said transducers to produce acoustic waves traveling along said path; and
an acoustic detector for receiving at least reflection signals from said region in response to said produced acoustic waves.

34. Apparatus according to claim 33 wherein said at least one transducer comprises a plurality of spaced transducers positioned along a straight line.

35. Apparatus according to claim 34 and including a plurality said linearly arrayed transducers, forming a planar array of transducers, said array providing coverage of a volume of the region.

36. Apparatus according to claim 26 and including a signal combiner for receiving said signals and combining said signals received from transducers oppositely placed on the path to provide acoustic impedance values along the path.
